(19) United States
(12) Patent Application Publication
(10) Pub. No.: US 2021/0358628 A1
(43) Pub. Date: Nov. 18, 2021

(54) DIGITAL COMPANION FOR HEALTHCARE

(71) Applicant: VALOTEC, Villejuif (FR)

(72) Inventors: Jean-Christophe LOURME, Villejuif (FR); Gary TENDON, Palaiseau (FR); Chloé ROLLAND, Villejuif (FR); Jeremie PINOTEAU, Le Kremlin-Bicêtre (FR)

(73) Assignee: VALOTEC, Villejuif (FR)

(21) Appl. No.: 17/282,152

(22) PCT Filed: Oct. 9, 2019
(86) PCT No.: PCT/EP2019/077347

§ 371 Date: Apr. 1, 2021

Related U.S. Application Data
(60) Provisional application No. 62/783,655, filed on Dec. 21, 2018.

(30) Foreign Application Priority Data
Oct. 9, 2018 (EP) 18306333.8

(51) Int. Cl. G16H 50/20; G16H 20/10

(52) U.S. Cl. CPC G16H 50/20; G16H 20/10

(57) ABSTRACT

A system for providing user motivating suggestion having a user interface; a processor; a HEP module for health evaluation and prescription of a user configured to provide a prescription of the basis of heath related information concerning the user; a PEM module for personality evaluation and user motivation, configured to perform an evaluation of the type of personality of the user and to identify the main driver of user motivation so as to select at least one category of services capable of motivate the user which is adapted to the prescription; a PAS module for user services configured to search from an available service included in the category of services and a PIC module for user interaction and context detection configured to

### FIELD OF INVENTION

The present invention pertains to the field of health-related services. In particular, the invention relates to a system designed for the evaluation of the health status of a user and for the output to the user of suggestions, recommendations or contents aiming to maintain or improve his health status.

### BACKGROUND OF INVENTION

Europe is facing huge demographic changes because the European population is ageing and life expectancy is increasing. Sustaining this ageing population requires an increasing focus on prolonging and achieving equity in good health and wellbeing throughout the life course. However, elderly people also increasingly require a package of long-term care that is partly delivered by healthcare and partly by social services, presenting a particular challenge for health systems.

Moreover, the percentage people suffering from chronic diseases increases with population aging and current lifestyles. The leading contributors to disease burden in older people are cardiovascular diseases, malignant neoplasm (cancer), chronic respiratory diseases, musculoskeletal diseases, mental and neurological disorders, diabetes mellitus, sensory impairments. Furthermore, elderly people live mostly alone and depend on caregivers, facing the risk of physical and psychological pain.

In this context, there is the need for developing innovative solutions to help patients manage their disease in order to maintain or improve their health, their independence and their safety.

The technological solution described herein offers the promise of addressing the improvement of health and well-being of patients.

### SUMMARY

The present invention relates to a module for health evaluation of a user and prescription, called HEP module, comprising:
a data storage medium configured to provide a health database to store health-related information concerning a user;
a computer readable medium comprising a program for health evaluation of a user and prescription comprising instructions executable by a processor, said instructions being configured to produce steps of:
   receiving health-related information of the user to be stored in the health database;
   generating a health output including a health status and/or a health trend of the user based on health-related information of the user, which is retrieved from the health database and which comprises physiological parameters for the evaluation of a health status of the user;
   evaluating the health status and/or the health trend of the user by comparing the health output to a predefined reference health target;
wherein, when the health output does not correspond to the predefined reference health target, the instructions are further configured to:
   extract, from the health-related user information, at least two relevant features representative of a symptomatic event;
   establish a correlation between the at least two relevant features representative of the symptomatic event;
   detect an occurrence pattern of the symptomatic event in at least one time period of predefined duration;
   using a classifier, determine for the user:
      at least one class associated to at least one type of user presenting the symptom corresponding to the symptomatic event, and
      a severity of said symptom,
   wherein the classifier receives as input the relevant features representative of the symptomatic event and the occurrence pattern; and
   generate a prescription of at least one action to be undertaken by the user on the basis of the class and the severity, such as taking a medication, reducing calorie intake, increasing physical activity.

According to one embodiment, the instructions are configured to control a wearable sensor of the user and/or to receive information related to the user from said wearable sensor of the user to be stored in the health database.

According to one embodiment, the instructions are configured to communicate the generated prescription to the user through a user interface, optionally after having sent the generated prescription to a medical staff for validation.

According to one embodiment, the instructions are further configured to emit an alert when the health output does not correspond to the predefined reference health target, notably communicated to a medical staff.

According to one embodiment, the instructions are further configured to obtain a feedback from the user in response to the generated prescription.

According to one embodiment, the instructions are further configured so that the generation of the prescription is obtained by means of a knowledge-based method.

The present invention further relates to a module for personality evaluation and user motivation, called PEM module, comprising:
a data storage medium configured to provide a user persona database to store personality-related information concerning a user comprising at least one main driver of user motivation;
a computer readable medium comprising a program for personality evaluation and user motivation comprising instructions executable by a processor, said instructions being configured to produce steps of:
   receiving as input a prescription of at least one action to be undertaken by the user;
   obtaining information concerning a type of personality associated to the user based on personality-related information of the user, which is retrieved from the user persona database;
   selecting at least one category of services on the basis of the prescription and the main driver of user motivation;
   determining adapted engagement strategy and motivation strategy for the user on the basis of the type of personality associated to the user and the prescription; and
   producing as output at least one service from the category of services conforming to said adapted engagement strategy and motivation strategy.

According to one embodiment, when more than one prescription is received as input, the instructions are further configured to produce a step of receiving as input a gravity index associated to each input prescription and giving higher priority to the category of services generated for the prescription associated to the highest gravity index.

According to one embodiment, the instructions are further configured so that the generation of the adapted engagement strategy and/or motivation strategy is obtained by means of a machine learning algorithm.

According to one embodiment, the output further comprises a predefined communication schedule, associated to the category of services, corresponding to at least one time at which at least one of the services of the selected category is executed.

According to one embodiment, the type of personality is computed by classifying the user into a class associated to a type of personality by means of a classifier receiving as input the personality-related information of the user.

The present invention further relates to a system for providing user motivating suggestion, comprising:
a processor;
a user interface;
a HEP module for health evaluation and prescription of a user according to any one of the embodiments described hereabove, the HEP module being configured to:
   receive as input health-related information of the user; and
   provide as output at least one prescription for the user;
a PEM module for personality evaluation and user motivation according to any one of the embodiments described hereabove; the PEM module being configured to:
   receive as input the prescription for the user generated by the HEP module and at least one compatible service generated by a PAS module for patient services, said compatible service satisfying the constraints imposed by the category of services produced by PEM module; and
   output at least one service and at least one instruction concerning the content of at least one message to deliver to the user;
a PAS module for patient services, comprising:
   a data storage medium configured to provide a service database to store information concerning services available to the user; and
   a computer readable medium comprising a program for user services comprising instructions executable by the processor, said instructions being configured to produce steps of receiving as input at least one category of services produced by the PEM module and searching in the service database at least one available service compatible to said category of services;
a PIC module for patient interaction and context detection comprising a computer readable medium comprising a program for user interaction and context detection comprising instructions executable by the processor, said instructions being configured to produce steps of:
   receiving as input at least a content of interaction with the user and the at least one instruction from the PEM module concerning the content of at least one message to deliver to the user; and
   controlling a dialog engine which uses content of interaction with the user and the instruction from the PEM module to generate messages electronically delivered to the user by means of the user interface;
wherein the system is configured to transfer information between the HEP module, PEM module, PAS module and PIC module.

According to one embodiment, the instructions of the PAS module are further configured to produce a step of identifying and listing the services in a geographical area, notably in proximity of the location of the user.

The present invention relates to a system for motivating people to do actions that contribute to improving or maintaining their physical health, cognitive health, mental health and social interactions.

A first aspect of the present invention relates to a module for health evaluation and prescription of a user, called HEP module, comprising:
a data storage medium configured to provide a health database to store health related information concerning a user;
a computer readable medium comprising a program for health evaluation of a user and prescription comprising instructions executable by a processor, said instructions being configured to:
   receive health related information of the user to be stored in the health database;
   access health related information of the user retrieved from the health database, the user information being relevant to evaluate the health status of the user;
   generate a health output concerning health status and/or health trend of the user based on the user information;
   evaluate the health status and/or health trend by comparing the health output to a predefined reference target;
wherein, when the health output does not correspond to the predefined reference target, the instructions are further configured to:
   extract relevant features from the user information, wherein at least one of the relevant features represents the occurrence of at least one symptomatic event;
   search for correlation between the relevant feature representing the symptomatic event and at least one other relevant feature;
   detect an occurrence pattern of the symptomatic event in at least one time period of predefined duration;
   using a classifier, classify the user into at least one class associated to at least one type of user presenting at least one symptom and a severity of said symptom, wherein the classifier receives as input at least one relevant feature representing the symptomatic event and the occurrence pattern; and
   generate at least one prescription for the user on the basis of the class and the severity.

This HEP module advantageously allows to detect the presence of symptomatic events and to correlate them with other relevant features, as done when following a medical reasoning. This correlation allows then to better classify the patient in order to obtain an effective prescription.

According to one embodiment, the instructions of the HEP module are further configured to control a wearable sensor of the user and/or to receive information related to the user from said wearable sensor of the user to be stored in the health database.

According to one embodiment, the instructions of the HEP module are further configured to communicate the generated prescription to the user through a user interface, optionally after having sent the generated prescription to a medical staff for validation.

According to one embodiment, the instructions of the HEP module are further configured to emit an alert when the health output does not correspond to the predefined reference target, notably communicated to a medical staff.

According to one embodiment, the instructions of the HEP module are further configured to obtain a feedback from the user in response to the generated prescription.

According to one embodiment, the instructions of the HEP module are further configured so that the generation of the prescription is obtained by means of a knowledge-based method.

The second aspect of the present invention relates to a module for personality evaluation and user motivation, called PEM module, comprising:
a data storage medium configured to provide a user persona database to store information concerning a user, said information concerning at least one main driver of user motivation;
a computer readable medium comprising a program for personality evaluation and user motivation comprising instructions executable by a processor, said instructions being configured to:
   receive as input at least one prescription for the user and the associated severity and the contents of potential interactions between the user and a dialog engine;
   access user information retrieved from the user persona database (201), the user information being relevant for the user personality evaluation;
   obtain information concerning a type of personality associated to the user;
   select at least one category of services on the basis of the prescription and the main driver of user motivation;
   determine an optimal engagement strategy for the user on the basis of the type of personality associated to the user;
   determine an optimal motivation strategy for the user the basis of the type of personality associated to the user and the prescription; and
   produce as output the at least one category of services.

According to one embodiment, when more than one prescription is received as input, the instructions are further configured to receive as input a severity associated to each input prescription and to give higher priority to the category of services generated for the prescription associated to the highest severity.

According to one embodiment, the instructions of the PEM module are further configured so that the generation of the optimal motivation strategy and/or the optimal engagement strategy is obtained by means of a machine learning algorithm.

According to one embodiment, the output further comprises a predefined communication schedule, associated to the category of services, corresponding to at least one time at which at least one of the services of the selected category is executed.

According to one embodiment, the type of personality is computed by classifying the user into a class associated to a type of personality by means of a classifier receiving as input the user information being relevant for the personality evaluation.

A third aspect of the present invention relates to a module for user services, called PAS module, comprising:
a data storage medium configured to provide a service database to store information concerning services available to the user; and
a computer readable medium comprising a program for user services comprising instructions executable by the processor, said instructions being configured to receive as input at least one category of services produced as output by the PEM module and search in the service database at least one service associated to said category of services.

According to one embodiment, the PAS module further comprises instructions configured to identify and list the services in a geographical area, notably in proximity of the effective location of the user.

A forth aspect of the present invention relates to a module for user interaction and context detection, called PIC module, comprising:
a computer readable medium comprising a program for user interaction and context detection comprising instructions executable by the processor, said instructions being configured to:
   receive as input at least a content of interaction with the user; and
   control a dialog engine which uses the at least one content of interaction with the user to generate messages electronically delivered to the user by means of the user interface.

A fifth aspect of the present invention relates to a system for providing user motivating suggestion, comprising:
a processor;
a user interface;
a HEP module for health evaluation and prescription of a user according to any one of the embodiments described hereabove, the HEP module being configured to:
   receive as input health related information of the user; and
   provide as output at least one prescription for the user;
a PEM module for personality evaluation and user motivation according to any one of the embodiments described hereabove; the PEM module being configured to:
   receive as input the prescription for the user generated by the HEP module and at least one compatible service generated by a PAS module for patient services, said compatible service satisfying the constraints imposed by the category of services produced by PEM module; and
   output at least one service and at least one instruction concerning the content of at least one message to deliver to the user;
a PAS module for patient services, comprising:
   a data storage medium configured to provide a service database to store information concerning services available to the user; and
   a computer readable medium comprising a program for user services comprising instructions executable by the processor, said instructions being configured to receive as input at least one category of services produced by the PEM module and search in the service database at least one available service compatible to said category of services;
a PIC module for patient interaction and context detection comprising a computer readable medium comprising a program for user interaction and context detection comprising instructions executable by the processor, said instructions being configured to:
   receive as input at least a content of interaction with the user and the at least one instruction from the PEM module (2) concerning the content of at least one message to deliver to the user; and
   control a dialog engine which uses the content of interaction with the user and the instruction from the PEM module (2) to generate messages electronically delivered to the user by means of the user interface;

wherein the system is configured to transfer information between the HEP module, PEM module, PAS module and PIC module.

Yet another aspect of the present invention relates to a method for health evaluation and prescription of a user, said method comprising the following steps:
receiving health related information of the user to be stored in a health database;
accessing health related information of the user retrieved from the health database, the user information being relevant to evaluate the health status of the user;
generating a health output concerning health status and/or health trend of the user based on the user information;
evaluating the health status and/or health trend by comparing the health output to a predefined reference target;

wherein, when the health output does not correspond to the predefined reference target, the instructions are further configured to:
extracting relevant features from the user information, wherein at least one of the relevant features represents the occurrence of at least one symptomatic event;
searching for correlation between the relevant feature representing the symptomatic event and at least one other relevant feature;
detecting an occurrence pattern of the symptomatic event in at least one time period of predefined duration;
by means of a classifier, classifying the user into at least one class associated to at least one type of user presenting at least one symptom and a severity of said symptom, wherein the classifier receives as input at least one relevant feature representing the symptomatic event and the occurrence pattern; and
generating at least one prescription for the user on the basis of the class and the severity.

Yet another aspect of the present invention relates to a method for personality evaluation and user motivation, said method comprising the following steps:
receiving as input at least one prescription for the user and an associated severity and the contents of potential interactions between the user and a dialog engine;
accessing user information retrieved from a user persona database, the user information being relevant for the user personality evaluation;
obtaining information concerning a type of personality associated to the user;
selecting at least one category of services on the basis of the prescription and the main driver of user motivation;
determining an optimal engagement strategy for the user on the basis of the type of personality associated to the user;
determining an optimal motivation strategy for the user the basis of the type of personality associated to the user and the prescription; and
producing as output the at least one category of service.

Yet another aspect of the present invention relates to a method for providing user motivating suggestion, said method comprising the steps of the method for health evaluation and prescription of a user according to the embodiments described hereabove, the steps of the method for personality evaluation and user motivation according to the embodiments described hereabove and following steps:
selecting at least one compatible service by searching in the service database, wherein said compatible service satisfies the constraints imposed by the category of services;
receive as input at least a content of interaction with the user;
defining at least one content for at least one message to deliver to the user using on the basis of the category of services and/or the at least one compatible service;
controlling a dialog engine so as to use content of interactions with the user and content for at least one message to generate messages electronically delivered to the user by means of the user interface.

According to one embodiment, the method further comprises a step consisting in the determination of a form on the basis of the optimal motivational strategy and optimal engagement strategy.

According to one embodiment, the method for health evaluation and prescription of a user and the method for personality evaluation and user motivation are computer implemented.

A further aspect of the present invention relates to a computer program comprising instructions which, when the program is executed by a computer, cause the computer to carry out the steps of the methods described hereabove.

Another aspect of the present invention relates to a computer readable storage medium comprising instructions which, when the program is executed by a computer, cause the computer to carry out the steps of the methods described hereabove.

### Definitions

“User” refers to a mammal, preferably a human. In the sense of the present invention, a subject may be a patient, i.e. a person receiving medical attention, undergoing or having underwent a medical treatment, or monitored for the development of a disease.
“Health status” refers to the health (good or poor) of a subject that may be assessed by multiple indicators concerning physical, psychological or mental conditions.
“Severity” refers to the extent of organ system derangement or physiologic decompensation for a patient.

### BRIEF DESCRIPTION OF THE DRAWINGS

FIG. 1 shows an example of HEP module for health evaluation and prescription of a user according to an embodiment of the present description.

FIG. 2 shows an example of PEM module for personality evaluation and user motivation according to an embodiment of the present description.

FIG. 3 shows an example of PAS module for patient services according to an embodiment of the present description.

FIG. 4 shows an example of PIC module for patient interaction and context detection according to an embodiment of the present description.

FIG. 5 shows an example of a system for providing user motivating suggestion comprising a HEP module for health evaluation and prescription of a user, a PEM module for personality evaluation and user motivation, a PAS module for patient services and PIC module for patient interaction and context detection according to an embodiment of the present description.

FIG. 6 shows an illustration of the processing steps taken to classify a patient according to one embodiment of the invention.

### DETAILED DESCRIPTION

HEP Module

A first aspect of the present invention relates to a module for health evaluation and prescription of a user, also called HEP module in the present description. The HEP module is configured to evaluate the global health status of the user and the follow up of said global health status so as to provide a prescription specific for the user aiming to improve his global health status.

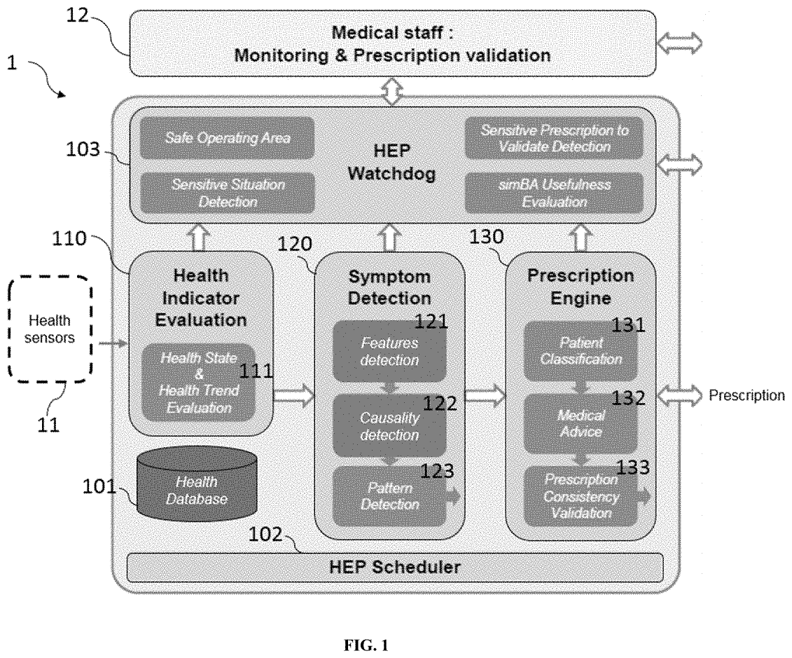

FIG. 1 illustrates a HEP computer module according to one embodiment of the present invention. As shown, the HEP module 1 comprises a data storage medium configured to provide a health database 101 to store health related information concerning the user. The health database 101 may be a secured electronic database stored in a mass storage device associated with a server system.

The HEP module 1 further comprises a computer readable medium comprising a program for health evaluation of a user and prescription comprising instructions executable by a processor. Said instructions may be organized in multiple blocks configured to communicate and cooperate with each other.

According to one embodiment, the instructions of HEP module 1 are configured to receive health related information of the user to be stored in the health database 101. In one embodiment, health related information concerning the user comprises information relating to at least one of the following: the demographic characterization of the subject (including age, gender, race, place of residence of the user, geographic travel history of the user, place of employment of the user, family unit of the user, hereditary disorders, etc.), the lifestyle history of the subject (such as for example body mass index, diet, alcohol, tobacco, and drug use, sexual history and habits, occupation, living conditions, etc.), the medical history of the subject (such as for example an earlier injury, hereditary disorders, earlier surgeries, etc.), health maintenance information (exercise habits, diet information, sleep data, vaccination data, therapy and counseling history), past medical history of the user, preexisting medical conditions of the user, current medications of the user, allergies of the user, surgical history, past medical screenings and procedures, past hospitalizations and visits and genetic profile of the user. Health related information may further comprise biometric data, including a variety of data sensed by one or more sensors 11 comprised into devices that may be worn by the user or IOT devices not worn directly on the user body, but arranged in physical proximity to the user. Said health related information further comprises medical device data collected from medical devices. Such medical device data may include, for example, inhaler usage data from an electronic inhaler device, blood sugar levels (or other physiological sugar levels) from an electronic blood sugar monitor, insulin pumping data from an electronic insulin pump, pulse oximetry data from an electronic pulse oximeter, weight data from a connected scale etc.

According to one embodiment, the HEP module instructions are configured to access health related information of the user retrieved from the health database 101, the user health related information being relevant to evaluate at least one aspect of the health status of the user.

According to one embodiment, the HEP module comprises instructions configured to control a wearable sensor of the user and/or to receive information related to the user from said wearable sensor of the user to be stored in the health database 101. In one example, the HEP module may control a wristband comprising a photoplethysmographic sensor to perform acquisition of the heart rate with a predefined frequency, i.e. each 2, 3, 4, 5 or 8 hours.

According to one embodiment, the HEP module 1 comprises instructions that are configured to generate a health output evaluating the health status and/or a health trend of the user based on the user health related information. In one embodiment, the health status is generated considering at least one or more indicators chosen from the following list: the physical status indicator, cognitive status indicator, mental status indicator or social status indicator. Said indicators may relate to absolute values or variation of a value of interest in the evaluation of the physical, cognitive, mental or social status of the user.

According to one embodiment, the health status and/or health trend are evaluated by comparing the health output to a predefined reference target. The predefined reference target may be a threshold representing a target that the health output should ideally reach or inversely that should not be exceeded. Alternatively, the predefined reference target may be a predefined reference range into which the health output should be comprised. In one example, health output generated is the average glycemia value that is compared to a predefined reference target being a range between 5.0 mmol/1 and 7.2 mmol/l (90-130 mg/dL) before meals, and less than 10 mmol/L (180 mg/dL) after meals (as measured by a blood glucose monitor).

The instructions described hereabove configured to receive and access health related information, generate health output and compare said health output to a predefined reference target are comprised in a block for health indicator evaluation 110.

The HEP module further comprises instructions configured to perform symptom detection, said instructions being optionally comprised in a block 120, said block being configured to communicate with the block 110.

In one embodiment, the block 110 is configured to transmit an order causing the execution of the instructions comprised in the block 120 when the health output does not correspond to the predefined reference target. In one example, the execution order is transmitted when the health output exceeds a threshold representing the predefined reference target or the health output is out of a range representing the predefined reference target.

In one embodiment, the block 120 is configured to receive as input the health-related information of the user retrieved from the health database 101, as consequence of the reception of the execution order from the block 110.

The symptoms detection is obtained by the execution of a succession of instructions comprised in block 120. According the illustrated embodiment of FIG. 1, the block 120 is configured to perform a feature detection 121 by extracting from data comprised in the health-related information at least one relevant feature. Said relevant features may be obtained for example from the analysis of the evolution of a biometric data over a predefined period of time, notably a pertinent feature may be a maximum, a minimum or the distance to a predefined threshold of the biometric data. According to one embodiment, at least one of the relevant features represents the occurrence of at least one symptomatic event. In one example, a first relevant feature is extracted from the analysis of blood sugar levels evolution during a predefined period of time as low blood sugar levels (i.e. below the 70 mg/dL threshold), corresponding to a hypoglycemia (i.e. relevant feature representing symptomatic event), while a second relevant feature is extracted from the level of physical activity of the user as a peak corresponding to a fitness session.

Other features detected may include: naps, nocturnal awakening, wakeup time, glycemia levels, rapid increase and decreases of glycemia levels. Distinguishing between complex data patterns, such as meals and snacking, may be done using classifiers, such as neural networks, decision trees etc., specifically trained/built on population data and/or user specific data.

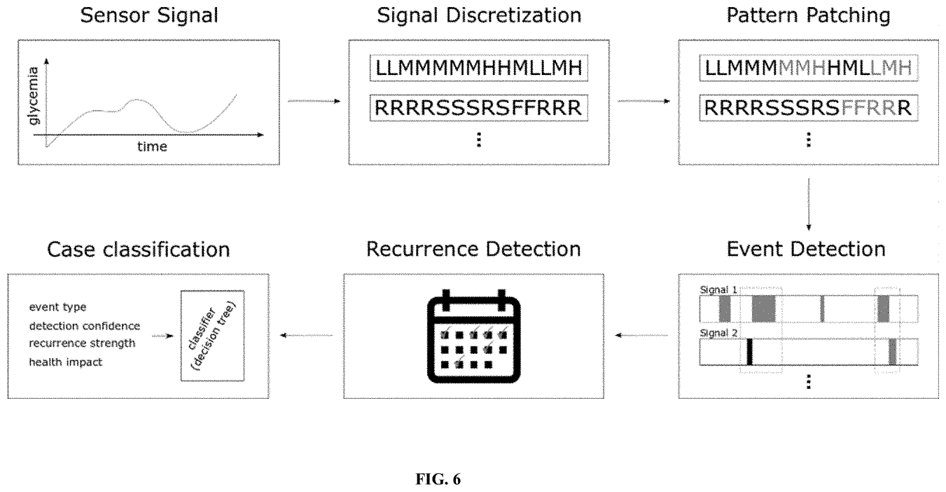

According to one embodiment illustrated in FIG. 6, the data signals acquired in a continuous mode from the sensors are simplified using a quantizer (i.e. data binning) and categorical signals are thereby created. A pattern matching algorithm may be then applied to the categorical signals for example using Hidden Markov Models. Similarly, a quantizer can be applied to different orders of derivatives of the sensor signals.

According to one embodiment, the block 120 comprises instructions 122 configured to detect a correlation and/or co-occurrence between at least one relevant feature representing at least one symptomatic event and at least one other relevant feature. In one example, the symptomatic event of hypoglycemia detected in the afternoon is associated to the relevant feature showing an intense physical activity. Or in another example, a symptomatic event of hyperglycemia detected in the afternoon is associated to relevant feature showing a lack of physical activity. The association between events can be detected by testing the independence between them for example using chi-square test, measuring their mutual information, or applying data mining techniques (itemset mining).

According to one embodiment, the block 120 comprises instructions 123 for pattern detection configured to detect the presence of a pattern in the occurrence of the symptomatic event in at least one time period of predefined duration. Said pattern detection allows to verify whether a symptomatic event is repeated over time and with which frequency, for example the hypoglycemia of the user occurred 3 times per week.

According to one embodiment, the frequency of occurrence of the symptomatic event is correlated with contextualized temporal information. In one example, a given symptomatic event is detected in 80% of the cases during the weekends.

The HEP module 1 comprises a set of instructions organized in a prescription engine block 130 which are configured to select at least one perception suitable for the user symptoms and health status. As shown by the illustrative embodiment in FIG. 1, the block 130 is configured to receive execution orders or information from the block 120.

Block 130 comprises instructions 131 configured to perform a classification of the user into at least one class associated to a type of user presenting at least one pattern of symptomatic event (i.e. symptom) occurrence and a severity of said symptom. The classifier receives as input at least one relevant feature representing a symptomatic event and at least one occurrence pattern. According to one embodiment, the classifier receives as input multiple relevant features, among which at least one represents a symptomatic event, and the occurrence pattern. According to this embodiment, the classes of the classifier are associated to at least one type of user presenting at least one symptom, a severity of said symptom and a type of recurrent behavior. Indeed, the analysis of relevant features may highlight recurrent behavior and habits of the user; for example, the analysis of the actimeter data provides the information on the rate of user physical activity and therefore deduce from this analysis whether or not the user is a sportive person. The classifier may further receive as input at least one correlation between the relevant feature representing the symptomatic event and at least one other relevant feature. In one example, a class may group all users performing low physical activity (e.g. lazy profile persons) and having hyperglycemia after meals. Block 130 further comprises instructions 132 to generate a prescription for the user on the basis of the class and the severity. According to one embodiment, the block 130 instructions are further configured so as to generate the prescription by means of a knowledge-based method. An example of prescription for a user showing hyperglycemia after lunch could be the reduction of calorie intake for lunch and/or an improvement of his physical activity.

According to one embodiment, the classifier is configured to classify the users of a class in different subclasses in order to provide a more detail description of the type of user.

When multiple prescriptions have been generated by instructions 132, each prescription is associated to a priority selected according to the severity of the symptoms. The block 130 may further comprise instructions 133 configured to verify the coherence between different prescriptions, and select the prescriptions associated to the highest priority.

For example, the prescription engine block 130 may comprise machine learning algorithms that, when executed, analyze data to determine one or more prescriptions for the user.

According to FIG. 1, the HEP module 1 further comprises a HEP watchdog block 103, comprising instructions configured to verify the correct functioning of the block 110, 120 and 130 described hereabove.

In one embodiment, HEP watchdog block 103 comprises instructions configured to detect sensitive situations that may degenerate in health complications for the user. According to one embodiment, the HEP module comprises instructions that are configured to emit an alert when the health output does not correspond to the predefined reference target, and notably communicate the alert to a medical staff. In block 103 instructions may be further configured to monitor the indicators of the health status in order to constantly verify whether or not their values are comprised in a functioning area of the HEP module. In one embodiment, the instructions of block 103 are configured to detect prescriptions sensitive for the user health and transmit them to a member of a medical staff for validation 12.

According to one embodiment, the instructions of the HEP module are further configured to communicate at least one generated prescription to the user through a user interface.

According to one embodiment, HEP watchdog block 103 further comprises instructions configured to monitor the evolution of the user health status and evaluate the usefulness of the prescriptions provided by HEP module 1 over a medium and long term. This is done by evaluating the rate of interactions of the user with the system and the evolution of health indicators computed by HEP. If for example, the application of the prescription of the HEP module does not produce an improvement of the indicators of the health status of the user an alert is sent to the user, to a member of a medical staff, to a relative of the user, or to any other qualified subject.

In the global architecture of the HEP module 1, as show in FIG. 1, the HEP schedule block 102 comprises instructions configured to coordinate the execution of the instructions comprised in the different blocks of the HEP module 1.

PEM Module

A second aspect of the present invention relates to a module for personality evaluation and user motivation, so-called PEM module in present description.

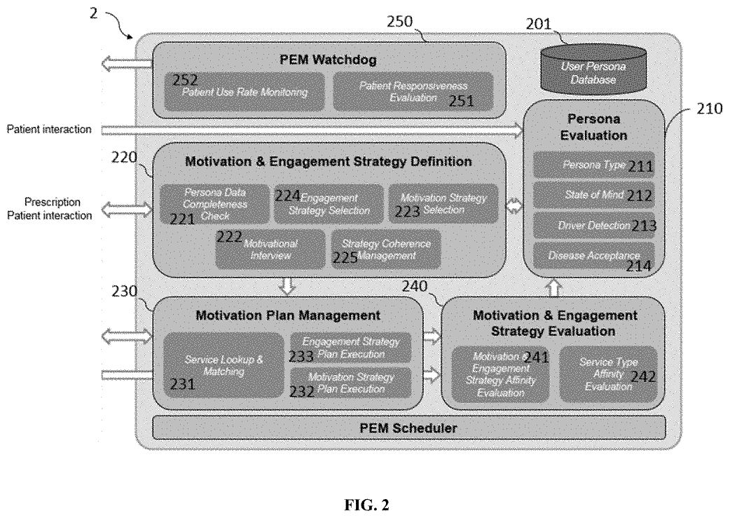

According to the embodiment illustrated in FIG. 2, the PEM module 2 comprises a data storage medium configured to provide a user persona database 201 to store information concerning a user, said information including at least one user preference and/or at least one personality model and/or one list of daily life activities comprising at least one predefined activity and/or at least one activity preselected by the user. The user may fill out a lifestyle questionnaire to obtain this information.

According to one embodiment, the PEM module 2 comprises a computer readable medium comprising a program for personality evaluation and user motivation comprising instructions executable by a processor.

Instructions comprised in PEM module 2 are grouped in an organized architecture of communicating blocks of instructions as shown in FIG. 2. Of course, as for instructions of HEP module 1, the instructions of PEM module 2 may be redistributed among the blocks still providing the desired outputs.

The PEM module is configured to transfer and receive information to other modules. In a preferred embodiment, PEM module is configured to exchange information with a dialog engine or a module comprising a dialog engine so as to exchange messages with the user.

According to one embodiment, the PEM module 2 comprises instructions configured to receive as input at least one prescription for the user and the contents of potential interactions between the user and a dialog engine. In one embodiment, PEM module instructions are configured to access user information retrieved from the user persona database 201, the user information being relevant for the user personality evaluation.

Said PEM module comprises a block 210 of instructions configured to provide a picture of the user personality and to follow the evolution of his state of mind at a medium and long term.

According to one embodiment, the persona evaluation block 210 comprises instructions 211 configured to obtain information concerning a type of personality of the user. Said information concerning a type of personality of the user may be stored user persona database 201.

In one embodiment, the PEM module comprises instructions so as to generate the type of personality by associating the user to a class representing a type of personality by means of a classifier, said classifier receiving as input the user information being relevant for the personality evaluation. A type of personality is a parameter characterized by a slow evolution over time therefore these classification instructions may be executed only the at a first use of the PEM module and the result of such a classification may be directly stored in the user persona database 201.

In one embodiment, the persona evaluation block 210 comprises instructions 212 is configured to detect a user mind status, preferably with a regular frequency so as to update user mind status for example every day, or every 2, 3, 4 day, or every 1, 2, 3 weeks. Said user mind status may be evaluated by combining multiple parameters concerning the user in a predefined period of time such as the frequency and intensity of physical activity, the frequency of social interactions, the correlation of relevant physiological parameters of the user during social interactions or work, or the like. The user may further provide a feedback concerning his feelings and emotions on a predefined period of time by using a dedicated rating scale.

In one embodiment, the persona evaluation block 210 comprises instructions 213 for the identification of the main driver of user motivation, such as at least one activity that is likely to interest the user, a center of interest and/or a goal that motivate the user to improve his health status. Instructions 213 may be configured to submit a survey to the user or receive as input information concerning the user preferred activities, goals and/or center of interests collected by a member of a medical staff. The information retrieved are stored in the persona database 201. For example, a user may have the goal of spending more time connecting with his family and playing with his grandchildren, travelling abroad, meeting new people (possibly creating a social circle of diabetic patients), developing a craft or hobby, practicing mindfulness and self-development, etc.

According to one embodiment, the PEM module comprises instructions configured to select at least one activity from the user persona database on the basis of the prescription.

In one embodiment, the persona evaluation block 210 comprises instructions 214 configured to evaluate the degree of illness acceptance in the user. This evaluation may be done via a survey to the patient, via inputs from medical staff, casual interactions of the module with the user, by means of a dialog engine (direct questions, roleplay game etc.) and/or weak signals in the interaction with the user. This information may be transmitted to a module for patient interaction and context detection capable of adapting the interactions with the user according to the degree of illness acceptance.

The persona evaluation block 210 is in communication with a motivation and engagement strategy definition block 220 configured to determine a motivation strategy and an engagement strategy for the user mainly on the basis of the type of personality associated to the user and the considered prescription. Concerning the example of the user presenting hyperglycemia after lunch for whom the prescription corresponds to a reduction of calories intake and/or a physical activity, the block 220 is designed to find at least one motivation strategy to induce the user to reduce his calories intake and/or increase his physical activity in the afternoon.

According to one embodiment, the block 220 is composed of multiple sub-blocks, all communicating with each other's and cooperating to the production of a block output.

According to the embodiment represented in FIG. 2, the block 220 comprises 5 sub-blocks: a persona data completeness sub-block 221, a motivation interview sub-block 222, an engagement strategy selection sub-block 223, motivation strategy selection sub-block 224 and strategy coherence management sub-block 225.

In one embodiment, sub-block 221 comprises instructions configured to verify that all information needed to produce a motivation strategy and an engagement strategy for the user are accessible to the block 220. In one example where sub-block 221 detects the absence of the information concerning the preferred activities, goals and/or center of interests necessary to develop a motivation strategy, the sub-block 221 may communicate with block 220 in order to obtain the execution of instructions 213 and obtain the missing information.

In one embodiment, sub-block 222 comprises instructions configured to transmit oral or vocal messages to the user, or to have an exchange with the user by means of a dialog engine. The transmitted messages use the main driver of user motivation so as to induce in the user a desire for change. In one example, sub-block 222 aims to persuade the user to have a small walk after lunch in order to have the possibility in the future to take his grandchildren to the play in a park.

In one embodiment, sub-block 223 comprises instructions configured to determine an optimal engagement strategy for the user based on at least one of the following: the type of personality, the mind status, the degree of illness acceptance or the main driver of user motivation. For example, for a user having an anxious type of personality, the sub-block 223 may select an engagement strategy consisting in fixing intermediate goals allowing to achieve a final goal that could otherwise seem unreachable and in providing to the user heartening and comforting messages by means of a dialog engine.

In one embodiment, sub-block 224 comprises instructions configured to determine an optimal motivation strategy for the user based at least on the type of personality and the prescription. The motivation strategy may consist in:
a negotiation with the user for a selection of a preferred strategy for addressing the health symptom, for example choosing between lower calories intake and increased physical activity;
a planification of the activity and its progression, for example providing a schedule of physical activity sessions proposing a progressively increasing time for the sessions on a month;
a communication of a message proposing the execution of an action and/or activity associated or not with supplementary information on the activity.

In one example, the sub-block 224 may select a motivation strategy consisting in sending every day a message suggesting to the user to have a walk after lunch and associating the message with a goal in terms of number of steps, which will increase progressively over time.

In one embodiment, sub-block 225 comprises instructions configured to verify the coherence between the motivation strategies and the engagement strategies selected, in the case when multiple prescriptions have been received. In this case, the motivation and engagement strategies associated to the prescription with the highest priority are selected first.

In one embodiment, information generated in block 220 (i.e. the activity, the motivation strategy and the engagement strategy, etc.) are transferred as input to a block for motivation plan management 230.

According to one embodiment, the PEM module instructions are further configured to generate the optimal motivation strategy and/or optimal engagement strategy by means of a machine learning algorithm, such as, but not limited to, neural networks, decision trees, k-nearest neighbor.

According to the embodiment illustrated in FIG. 2, block 230 comprises instructions 231 configured to perform the selection of at least one service or category of services adapted to the selected motivation strategy and selected activity interesting the user. In this embodiment, block 230 further comprises instructions 232 configured for the execution of a plan according to the motivation strategy. More in details, instructions 232 are configured to coordinate and organize the services chosen. In one example, the instructions 232 implement the transmission to the user of messages concerning the activities to do, such as daily notifications after lunch to suggest the user to have a walk after lunch, where the notification may further comprise a goal for each day. In this embodiment, block 230 comprises instructions 233 configured to execute a plan according to the engagement strategy. Indeed, instructions 233 are configured to run the different services which should reinforce user engagement. In one example, instructions 233 produces notifications to encourage the user to achieve his goal and congratulate him when the goal is achieved.

According to one embodiment, the PEM module further comprises information configured to generate and output a predefined communication schedule associated to the user motivation suggestion corresponding to at least one time at which the user motivation suggestion has to be communicated to the user.

Block 230 for motivation plan management is configured to communicate with block 240 for the motivation strategy evaluation. According to one embodiment, block 240 comprises instructions 241 configured to evaluate the efficacy of the motivation strategy and engagement strategy on the user by evaluating the frequency of use of a certain category of services, when the plan according to the motivation strategy and engagement strategy are executed. In one embodiment, block 240 comprises instructions 242 configured to evaluated the affinity and responsiveness of the user to the different categories of services proposed. The information obtained from block 240 may be transferred to the persona evaluation block 210.

The PEM module 2 further comprises a PEM watchdog block 250 comprising two sub-blocks 251 and 252. In one embodiment, sub-block 251 comprises instructions configured to detect possible changes in the user behavior during his interactions with the dialog engine communicating with the PEM module. This function may help in dealing with possible alerts. The instructions of sub-block 251 further run corrective actions in case of detection of lack of use of the PEM module, for example by sending a notification to a member of the medical staff or by sending a message to the user through the dialog engine. In one embodiment, sub-block 252 comprises instructions configured to monitor the use of the PEM module in order to ensure the adequacy of the PEM module for the user.

The PEM module 2 further comprises a PEM schedule block 202 comprising instructions configured to coordinated the execution of the different blocks of the PEM module.

PAS Module

A third aspect of the present invention concerns a module for patient services, also called PAS module in the following description.

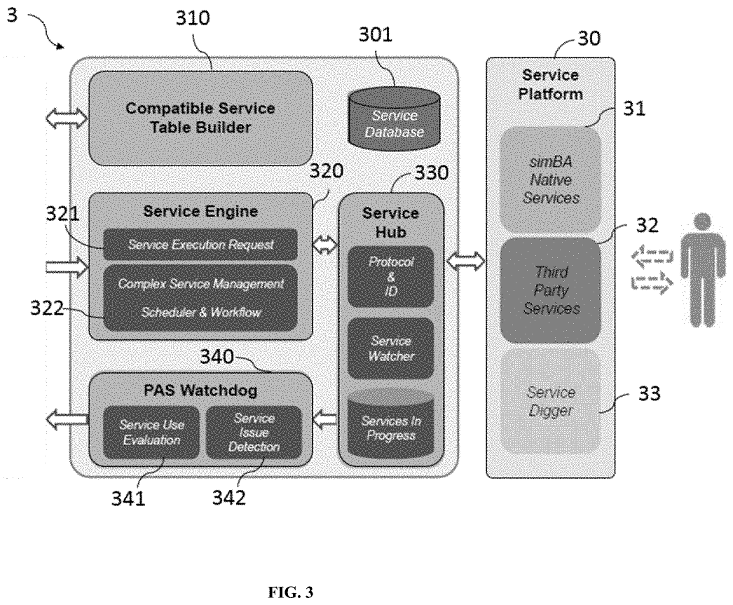

FIG. 3 illustrates a PAS computer module according to one embodiment of the present invention. As shown, the PAS module 3 comprises a data storage medium configured to provide a service database 301 to store information concerning services related to daily life activities.

According to one embodiment, the PAS module 3 comprises a computer readable medium comprising a program for user services comprising instructions executable by the processor, said instructions being configured to receive as input the category of services produced by the PEM module and search in the service database at least one service associated to said class of services.

The PAS module 3 further comprises a service platform 30 comprising the ensemble of services available by the PAS module. According to one embodiment, the service platform 30 comprises native services developed for the PAS module or systems comprising PAS module 31 or third parties' services available to the PAS module 32 such as for example navigations services (i.e. google maps). The service platform may further comprise service digger instructions 33 to perform a research of information on the web.

According to one embodiment, the PAS module 3 comprises multiple blocks of instructions cooperating with each other's. According to one embodiment, the PAS module 3 comprises instructions configured to receive as input:
at least a category of services for the user selected by a module for the personality evaluation and user motivation; and optionally;
a predefined communication schedule associated to the category of services corresponding to at least one time at which at least one of the services of the selected category should be executed.

According to one embodiment, PAS module 3 comprises a compatible service table builder block 310 comprising instructions configured to review the references, descriptions and constraints of the available services that satisfies the constraints imposed by category of services received as input.

According to one embodiment, PAS module 3 comprises a service engine block 320 having a service execution request sub-block 321 and a complex service management sub-block 322. In one embodiment, the sub-block 321 comprises instructions configured to control the execution of at least one of the services of the category in accordance with the predefined communication schedule so as to execute a desired service at a desired time. For example, a notification may be sent every day to the patient after lunch time as a reminder for doing a physical activity in the afternoon. In one embodiment, the sub-block 322 manages complex services composed of a succession or a combination of elementary services selected from the service platform 30, said complex services being chosen according to the input service category. In one example, a category of services may concern the calories intake and meals, and the complex service may be the preparation of a low calories meal. This complex service may comprise the selection of a recipe from a service providing multiple low calories meal recipes associated to an on-line service for ordering the necessary ingredients.

According to one embodiment, the PAS module comprises instructions configured to identify and list the services in a geographical area, notably in proximity to the effective location of the user.

The PAS module 3 further comprises a service Hub block 330 having the function of interface between the PAS module, or a system comprising the PAS module, and the services of the services platform 30. According to one embodiment, the service hub comprises a protocol and ID sub-block configured to centralize and manage the username and the information necessary to the access to the different services of the service platform 30. According to one embodiment, the service hub 330 further comprises a service watcher sub-block for the monitoring of the execution, and when possible, the use of the executed services in order to transmit the information to a PAS watchdog block 340.

The PAS module 3 further comprises a PAS watchdog block 340 configured to organize and control the execution of the instructions of the PAS module. According to one embodiment, the PAS watchdog block 340 comprises a sub-block for the service evaluation 341 and a sub-block for the service issue detection 342. In one embodiment, the sub-block 341 comprises instructions configured to detect that the proposed service have been effectively used by the user, for example that the user have seen a proposed video. In one embodiment, the sub-block 341 comprises instructions configured to detect and communicate the presence of a possible problem, for example the proposed video was not visualized due to a connection issue.

PIC Module

A fourth aspect of the present invention relates to a module for patient interaction and context detection, also called PIC module in the following description.

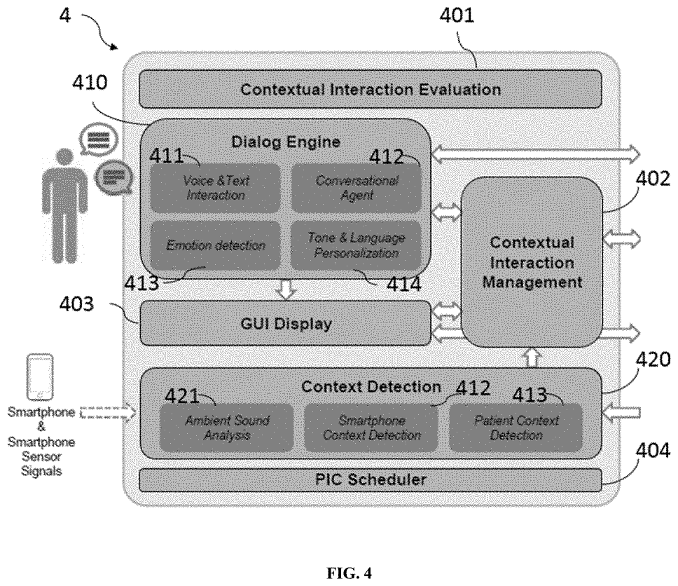

FIG. 4 illustrates a PIC computer module according to one embodiment of the present invention. The PIC module 4 comprises a computer readable medium comprising a program for patient interaction and context detection comprising instructions executable by the processor. According to one embodiment, the PIC module exchanges data with other computer modules.

According to one embodiment, the PIC module 4 is configured to receive as input at least a content of interaction with the user. The PIC module 4 may further receive as input the type of personality and state of mind of the user.

As shown in FIG. 4, the PIC module 4 comprises a contextual interaction evaluation block of instructions 401 configured to evaluate the effectiveness of the interactions between the PIC module 4 and the user, globally and as a function of a detected context. Such evaluation may be done for example on the basis of the number of answers of the user.

The PIC module 4 comprises a dialog engine block 410 which manages the production of messages and the reception and interpretation of the user answers. According to one embodiment, the dialog engine uses at least a content of interaction with the user to generate messages destined to be electronically delivered to the user by means of the user interface.

In one embodiment, the PIC module comprises the following four sub-blocks configured to cooperate:
voice and text interaction sub-block 411, comprising instructions configured to manage the interface with the user by reception of written or oral messages;
conversational agent sub-block 412, comprising instructions configured to communicate information to the user and to manage the flow of information provided by the user through massages in order to retrieve the necessary information;
emotion detection sub-block 413 comprising instructions configured to detect emotions of the user by means of analysis of the user written or oral messages;
tone and language personalization sub-block 414 comprising instructions configured to generate messages for the user wherein the form, the tone and the vocabulary is chosen accordingly to the detected context, the object of the message, the type of personality and state of mind.

The PIC module 4 further comprises a contextual interaction module 402 configured to manage the request for interaction and access of the user depending on the context and the history of the interaction.

The PIC module 4 comprises a GUI display block 403 configured to display information, that may be provided by another module communicating with the PIC module.

The PIC module 4 further comprises a context detection block 420, comprising instruction configured to detect in real-time the global context in which is situated the user. According to one embodiment, the detection block 420 comprises an ambient sound analysis sub-block 421 configured to analyses the auditory information in the environment of the user (e.g. if the patient is in a crowded place). According to one embodiment, the detection block 420 further comprises a smartphone context detection sub-block 422 configured to analyze the information on the user smartphone (e.g. app usage) and a patient context detection sub-block 423 configured to analyze data from at least a sensor present in the environment of the user (e.g. GPS or gyroscope).

The PIC module comprises a PIC schedule 404 configured to manage the execution of the different blocks of the PIC module 4.

System

A fifth aspect of the present invention relates to a system for providing user motivating suggestion comprising a processor, a user interface, a HEP module 1, a PEM module 2, a PAS module 3 and a PIC module 4.

According to one embodiment, the system 5 comprises a HEP module for health evaluation and prescription of a user according to the embodiments described hereabove.

According to one embodiment, the system 5 further comprises a PEM module for personality evaluation and user motivation of a user according to the embodiments described hereabove.

According to one embodiment, the system 5 further comprises a PAS module 3 for patient services according to the embodiments described hereabove.

According to one embodiment, the system 5 further comprises a PIC module 4 for patient interaction and context detection according to the embodiments described hereabove.

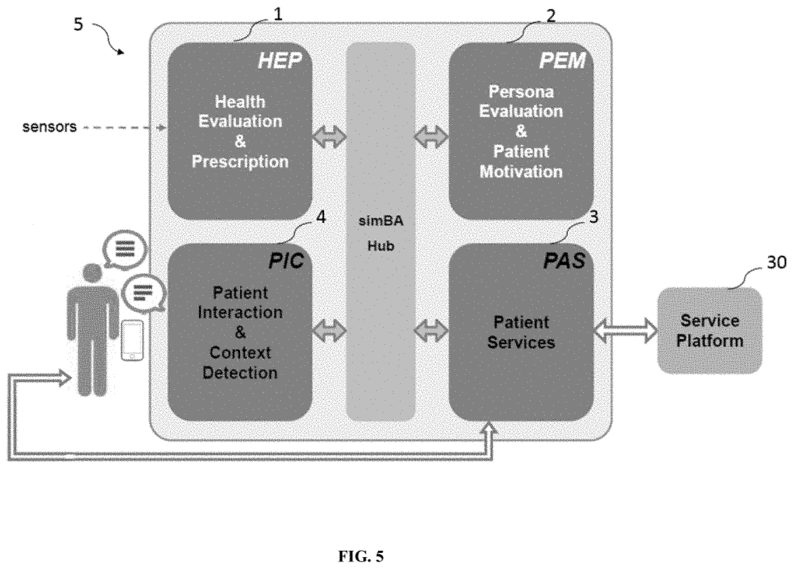

FIG. 5 illustrates a system according to one embodiment of the present invention. As show, the four modules are in communication with each other. According to one embodiment, the system 5 is a computer system comprising at least a processor and a user interface that are detailed later the description.

According to one embodiment, the HEP module 1 is configured to receive health related information of the user. Said health related information may be obtained from an input received by at least one health wearable sensor 11, from the access to the health database 101 or by an interaction of the PIC module 4 with the user.

According to one embodiment, the HEP module 1 is configured to provide as output at least one prescription for the user. In one embodiment, the HEP module further provides as output the severity of the symptoms associated to the prescription.

According to one embodiment, the PEM module 2 is configured to receive as input the prescription for the user generated by the HEP module 1 and optionally the severity of the symptoms associated to the prescription.

According to one embodiment, the PEM module 2 is configured to generate as output at least one category of services.

According to one embodiment, the PAS module 3 is configured to receive as input at least one category of services, produced as output by the PEM module 1, and search in the service database 301 at least one available service associated to said category of services.

According to one embodiment, the PEM module 2 transfers the category of services to the PAS module 3 in order to select at least one service compatible with the constraints imposed by the category of services. Said at least one compatible service is then transfer back to the PEM module 2.

According to one embodiment, the PIC module 4 is configured to generate as output messages electronically delivered to the user by means of the user interface.

According to one embodiment, the PEM module 2 defines the content of at least one message according to the least one compatible service and output at least one instruction for the generation of a message to deliver to the user concerning said content. Said instruction is used in the PIC module 4 to control the dialog engine 410 to generate a message delivered to the user.

According to one embodiment, the optimal engagement strategy and/or the optimal motivation strategy produced by the PEM module 2 are used to define the best type and the form of the interaction to utilize with the user. This information is used in combination with the PIC module 4 so as to adapt the interaction with the user to his personality and actual state of mind. For example, the optimal engagement strategy and/or the optimal motivation strategy may suggest that the type of interaction should be a negotiation and the form of the interaction should be positive or should comprise explications.

According to one embodiment, the block 220 of the PEM module 2 generates instructions defining the content of a message and the type and the form of the interaction to provide to the PIC module 4 in a phase of exchange with the user aiming to propose the compatible services selected and allow the user to choose at least one of them.

According to one embodiment, the PEM module 2 is configured to send instructions for the generation of a message to the PIC module 4 during the execution of the personality evaluation block 220 in order generate an interaction between the system 5 and the user so as to obtain for example information on the user actual mood.

According to one embodiment, the PEM module 2 is configured to send instructions for the generation of a message to the PIC module 4 during the execution of the block 240 in order generate an interaction between the system 5 and the user so as to obtain a feedback on the user evaluation of the compatible services proposed to the user.

The computer system may be embodied as a computing device, providing operations according to the instructions of a HEP module 1, a PEM module 2, a PAS module 3 and a PIC module 4, or any other processing or computing platform or component described or referred to herein. In alternative embodiments, the computer system operates as a standalone device or may be connected (e.g., networked) to other machines. The computer system may be a personal computer (PC) that may or may not be portable (e.g., a notebook or a netbook), a tablet, a set-top box (STB), a gaming console, a Personal Digital Assistant (PDA), a mobile telephone or smartphone, a web appliance, a network router, switch or bridge, or any machine capable of executing instructions (sequential or otherwise) that specify actions to be taken by that machine.

According to one example, the computer system includes a processor (e.g., a central processing unit (CPU), a graphics processing unit (GPU) or both), a main memory and a static memory, which communicate with each other via an interconnect (e.g., a link, a bus, etc.). The computer system further includes a user interface that may comprise a video display unit, an alphanumeric input device (e.g., a keyboard), and a user interface (UI) navigation device (e.g., a mouse). In one embodiment, the video display unit, input device and UI navigation device are a touch screen display. The computer system may additionally include a storage device (e.g., a drive unit), a signal generation device (e.g., a speaker), an output controller, a power management controller, and a network interface device (which may include or operably communicate with one or more antennas, transceivers, or other wireless communications hardware), and one or more sensors, such as a GPS sensor, compass, location sensor, accelerometer, or other sensor.

The storage device includes a machine-readable medium on which is stored one or more sets of databases and instructions (e.g., software) embodying or utilized by any one or more of the functions described herein. The instructions may also reside, completely or at least partially, within the main memory, static memory, and/or within the processor during execution thereof by the computer system, with the main memory, static memory, and the processor also constituting machine-readable media.

The instructions may further be transmitted or received over a communications network using a transmission medium via the network interface device utilizing any one of a number of well-known transfer protocols (e.g., HTTP). Examples of communication networks include a local area network (LAN), wide area network (WAN), the Internet, mobile telephone networks, Plain Old Telephone (POTS) networks, and wireless data networks (e.g., Wi-Fi, 3G, and 4G LTE/LTE-A or WiMAX networks). The term “transmission medium” shall be taken to include any intangible medium that is capable of storing, encoding, or carrying instructions for execution by the machine, and includes digital or analog communications signals or other intangible medium to facilitate communication of such software.

Embodiments comprised in the present description may also be implemented as instructions stored on a computer-readable storage device or storage medium, which may be read and executed by at least one processor to perform the operations described herein. A computer-readable storage device or storage medium may include any non-transitory mechanism for storing information in a form readable by a computer. For example, a computer-readable storage device or storage medium may include read-only memory (ROM), random-access memory (RAM), magnetic disk storage media, optical storage media, flash-memory devices, and other storage devices and media. In some embodiments, the electronic devices and computing systems described herein may include one or more processors and may be configured with instructions stored on a computer-readable storage device.

Embodiments and examples, as described herein, may include, or may operate on, logic or a number of components, modules, or mechanisms. Modules are tangible entities (e.g., hardware) capable of performing specified operations and may be configured or arranged in a certain manner. In an example, circuits may be arranged (e.g., internally or with respect to external entities such as other circuits) in a specified manner as a module. In an example, the whole or part of one or more computer systems (e.g., a standalone, client or server computer system) or one or more hardware processors may be configured by firmware or software (e.g., instructions, an application portion, or an application) as a module that operates to perform specified operations. In an example, the software may reside on a computer readable medium. In an example, the software, when executed by the underlying hardware of the module, causes the hardware to perform the specified operations.

While various embodiments have been described and illustrated, the detailed description is not to be construed as being limited hereto. Various modifications can be made to the embodiments by those skilled in the art without departing from the true spirit and scope of the disclosure as defined by the claims.

### EXAMPLES

The present invention is further illustrated by the following examples.

### Example 1

This example provides a description of the steps performed by the system 5 for the case wherein the user is a patient having type 2 diabetes. In this example the patient uses insulin mono-injection and realizes his insulin injections in the evening.

The first step is performed by block for health indicator evaluation 110 of HEP module 1, where the health output is calculated from the user information stored in the health database 101 and comprises the time in range, defined as a measure of time where the blood glucose remains within the proposed target range, and the average glycemia.

The second step, performed by the HEP module 1 watchdog block 103, comprises the verification that the health output is not in a critical range of values and that the health status is coherent with the use of the system 5. In this example, the system 5 is configured to work for type 2 diabetic patient and the health status of the patient is improved in the last 3 months therefore the health output is not in a critical range.

The third step, performed in the block symptom detection 120 of the HEP module 1, consists in:
extracting a first relevant feature from the user information by analyzing the continuous glucose measurements as function of time (i.e. calculating the area under the curve of the glycemia) in order to detect the occurrence of the symptomatic event of hyperglycemia in the early afternoon;
extracting a second relevant feature concerning food intake by analyzing the signal of an actimeter worn by the patient and the time, and a rise in glycemia levels in order to detect that the patient had lunch at mid-day;
extracting a third relevant feature concerning the intensity of the physical activity performed by the patient as function of time using the actimetry signal so to detect an absence of physical activity in the afternoon;
deducing, by correlation between the relevant features, that the hyperglycemia follows calories intake of the lunch; and
detecting an occurrence pattern of the hyperglycemia, by measuring, that 80% of days, lunch is followed by hyperglycemia.

The fifth step performed in the prescription engine of block 130 of the HEP module 1, consists in the association of the patient to the class of patient having a too important intake of calories which is not compensated by physical activity and as consequence the prescription of a change of means in order to reduce the calories intake and/or the performance of a physical activity in the afternoon.

The firth step, implemented by the HEP watchdog block 103 of the HEP module 1, verifies that the prescriptions are not dangerous for the patient and that in this case there is no need of have the validation of the prescriptions by a member of the medical staff.

The sixth step is performed by the block 210 of the PEM module 2, where the information concerning the type of personality of the patient is retrieved by the persona database 201 since in this example this information had been already calculated and stored in the user persona database 201. In this example, the patient is associated to one class of type of personality comprising introvert and serious persons.

The seventh step is performed by the motivation and engagement strategy definition block 220. This step consists in:
verifying that the user persona database comprises enough updated information concerning the type of patient, his mind status and his main driver of motivation;
selecting the optimal engagement strategy and the optimal motivation strategy on the basis of the knowledge that the patient is introvert and serious; and
verifying that the selected engagement strategy and motivation strategy are not in conflict with other ongoing routines.

In this example the user's mind status is relaxed, notably regarding his diabetes, and the main driver of motivation consists in offering clear opportunities of self-improvement.

In this example, the optimal engagement strategy and optimal motivation strategy consists in choosing to provide to the patient explanations concerning the effects of hyperglycemia on his global health and then allowing the patient to choose at least one of two possible objectives: a reduction of calories intake and/or an improved physical activity.

The eighth step of detecting whether or not the patient in awaken, engaged in a conversation or using his cellphone is performed by the context detection block 420 of the PIC module.

The ninth step, performed by the contextual interaction management block 402, consists in giving the authorization to send a message to the patient since according to step eight his is awake and not busy.

The tenth step consists in the communication with the patient in order to finalize the choice of the objective, where the PIC module manage the communication with the patient and the PEM module manage the negotiation concerning the objective. In this example, the patient decides to gather unchanged his nutritional habits but to increase the physical activity. Therefore, in this example the category of services associated to the objective chosen by the patient concerns the physical activity.

The eleventh step, performed by block 230 of the PEM module 2, consists in starting a research for available services using the PAS module.

The twelfth step is performed in the block 310 of the PAS module 3 and consists in the research in the database and service platform 30 all the services comprised in the chosen category of services and compatible with other patient constraint, such as his geographical localization, his budget, possible contraindication, the presence of public transport etc. The block 310 send the information concerning the reference, description and constraints of he services compatible with the request to the PEM module 1. The services selected may be:
the suggestion of informative videos requiring a quite environment to be seen (i.e. constraint);
a walk in a park having as constraint the good weather, proximity of the patient to the park and the present of no contraindication for the patient to do a walk;
fitness exercises at home having as constraint that the patient has to be at home;
a fitness gym proposing a soft gym session in the afternoon having as constraint the proximity of the patient the fitness gym the present of no contraindication for the patient to do the proposed exercises.

The thirteenth step is implemented in the PEM module 2 by the block 230 and consists in the validation the type of personality of the patient matches with the proposed services, perform a reservation of the service for a suitable date and time in the day according to the occurrence pattern of the hyperglycemia of the patient and define a communication schedule for the reminder and motivational messages to be sent to the patient.

In the case when the service to provide to the user is the walk in park, the fourteenth step consists in the:
reception of command for the execution of the service associated to the walk in a park;
according to the time in the communication schedule, send a message to the user comprising the suggestion of having a walk in the park in proximity to his residence, said message demanding validation of the activity if the patient wants to perform the walk;
after patient validation, starting the GPS navigator application in order to propose a path to reach the park; and
when a predefined distance is detected between the patient and the park, sending a message to receive a feedback on the activity;
   said fourteenth step being performed in PAS module 3 by the service engine block 320.

The fifteenth step is performed by the PAS watchdog block 340 to verify whether or not the patient went to the park by using the GPS application and whether or not the patient answered to notifications or messages.

The sixteenth step is performed by the block 240 of PEM module 2 and comprises:
retrieving the information concerning the proposed services (i.e. whether or not the patient performed and liked them);
if the patient never used the service of the fitness gym or fitness exercises, PEM module reduces the weight of the physical activity as driver for the user motivation in the user persona database;
if the negotiated plan is globally followed by the patient, the user persona database is updated in order to reflect the affinity of the patient to this strategy.

The seventeenth step is performed by the PEM watchdog block 250 in order to verify if the frequency of use of the system 5 is above a predefine threshold and whether an important change in patient habits in the utilization of the system has occurred.

### Example 2

This example provides a description of the steps performed by the system 5 for the case wherein the user is a patient that needs to monitor heart palpitations and extrasystole events. In this example the patient is equipped with an ECG sensor and an activity tracker.

The first step is performed by block for health indicator evaluation 110 of HEP module 1, where the health output is calculated from the user information stored in the health database 101 and comprises the mean heart rate and the heart rate variability.

The second step, performed by the HEP module 1 watchdog block 103, comprises the verification that the health output is not comprised in a critical range of values and that the health status is coherent with the use of the system 5. In this example, the system 5 is configured to work for patients presenting a heart rate variability of SDNN>50 ms (a low heart rate variability may be symptomatic of other heart diseases).

The third step, performed in the block symptom detection 120 of the HEP module 1, consists in:
extracting a first relevant feature from the user information by analyzing the ECG in order to detect the occurrence of an extrasystole event, using a classifier which considers the shape of the measured QRS complex, T wave and P wave;
extracting a second relevant feature concerning the presence of a work meeting that day using the user's smartphone calendar;
extracting a third relevant feature concerning the intensity of the physical activity performed by the patient as function of time using the actimetry signal so to detect an absence of physical activity in the afternoon;
deducing, by correlation between the relevant features, that the extrasystole event did not follow an intense physical activity but preceded a work meeting; and
detecting an occurrence pattern, by measuring, that every week on Thursdays, multiple extrasystole events precede the user's work meetings.

The fourth step performed in the prescription engine of block 130 of the HEP module 1, consists in the association of the patient to the class of patients having heart palpitations linked to work stress.

The fifth step, implemented by the HEP watchdog block 103 of the HEP module 1, verifies that the prescriptions are not dangerous for the patient and that in this case there is no need of having the validation of the prescriptions by a member of the medical staff.

The sixth step is performed by the block 210 of the PEM module 2, where the information concerning the type of personality of the patient is retrieved by the persona database 201 since in this example this information had been already calculated and stored in the user persona database 201. In this example, the patient is associated to a class of type of personality comprising of introvert and open-minded persons.

The seventh step is performed by the motivation and engagement strategy definition block 220. This step consists in:
verifying that the user persona database comprises enough updated information concerning the type of patient, his mind status and his main driver of motivation;
selecting the optimal engagement strategy and the optimal motivation strategy on the basis of the knowledge that the patient is introvert and serious; and
verifying that the selected engagement strategy and motivation strategy are not in conflict with other ongoing routines.

In this example the user is suffering from mild depression (i.e. state of mind) and the main driver of motivation is reducing anxiety.

In this example, the optimal engagement strategy and optimal motivation strategy consists in choosing to provide to the patient relaxing breathing exercises or a light physical activity before the stressful event linked to the detected extrasystole events.

The eighth step of detecting whether or not the patient is awake, engaged in a conversation or using his cellphone is performed by the context detection block 420 of the PIC module.

The ninth step, performed by the contextual interaction management block 402, consists in giving the authorization to send a message to the patient since according to step eight his is awake and not busy.

The tenth step consists in the communication with the patient in order to finalize the choice of the objective, where the PIC module manage the communication with the patient and the PEM module manage the negotiation concerning the objective. In this example, the patient decides to perform a relaxing breathing exercise instead of the light physical activity.

The eleventh step, performed by block 230 of the PEM module 2, consists in starting a research for available services using the PAS module.

The twelfth step is performed in the block 310 of the PAS module 3 and consists in the research in the database and service platform 30 all the services comprised in the chosen category of services and compatible with other patient constraint, such as his geographical localization, his budget, possible contraindications, the presence of public transport etc. The block 310 send the information concerning the reference, description and constraints of the services compatible with the request to the PEM module 1. The services selected may be:
the suggestion of informative videos requiring a quite environment to be seen (i.e. constraint),
a guided meditation session through a dedicated application,
a sophrology course available nearby and the like.

The thirteenth step is implemented in the PEM module 2 by the block 230 and consists in validating whether the type of personality of the patient matches with the proposed services, perform a reservation of the service for a suitable date and time in the day according to the occurrence pattern and define a communication schedule for the reminder and motivational messages to be sent to the patient. The reminder may include an encouraging quote such as: Don't forget your meditation session today. As St Francis de Sales said: “Where there is peace and meditation, there is neither anxiety nor doubt.”.

In the case when the service to provide to the user is a guided meditation session through a dedicated application, the fourteenth step consists in the:
reception of command for the execution of the service associated to the meditation session;
according to the time in the communication schedule, send a message to the user comprising the suggestion of having a meditation session, said message demanding validation of the activity from the user;
after patient validation, starting the meditation application in order to guide the meditation session; and
when a predefined duration of use of the meditation sessions is detected, sending a message to receive a feedback on the activity;
   said fourteenth step being performed in PAS module 3 by the service engine block 320.

The fifteenth step is performed by the PAS watchdog block 340 to verify whether or not the patient performed the meditation session and whether or not the patient answered to notifications or messages.

The sixteenth step is performed by the block 240 of PEM module 2 and comprises:
retrieving the information concerning the proposed services (i.e. whether or not the patient performed and liked them);
if the patient never used the service of the informative video or the sophrology class, PEM module reduces the weight of the activity class as driver for the user motivation in the user persona database;
if the negotiated plan is globally followed by the patient, the user persona database is updated in order to reflect the affinity of the patient to this strategy and increase the weight of the activity class as driver for the user motivation in the user persona database.

The seventeenth step is performed by the PEM watchdog block 250 in order to verify if the frequency of use of the system 5 is above a predefine threshold and whether an important change in patient habits in the utilization of the system has occurred.

1-13. (canceled)

14. A system for health evaluation of a user and prescription comprising:
at least one input adapted to receive overtime health-related information of the user;
at least one processor configured to, iteratively:
monitor at least one relevant feature representative of a symptomatic event and at least one other relevant feature, being a behavioral event;
establish a correlation between said at least one relevant feature representative of a symptomatic event and said at least one behavioral event;
detect in at least one time period of predefined duration an occurrence pattern of the symptomatic event;
use a classifier to determine for the user at least one class associated to at least one type of user presenting a symptom corresponding to a symptomatic event and associated to a recurrent behavior, wherein the classifier receives as input the relevant features representative of the symptomatic event, the at least one behavioral event, the correlation and the occurrence pattern; and
generate a prescription of at least one action to be undertaken by the user on the basis of the class so as to improve his health; and
at least one output adapted to provide said prescription.

15. The system according to claim 14, wherein establishing said correlation from causality detection from the at least one behavioral event to the at least one relevant feature representative of a symptomatic event.

16. The system according to claim 14, wherein the occurrence pattern of the symptomatic event is correlated with contextualized temporal information.

17. The system according to claim 14, wherein the health-related information comprises biometric data sensed by at least one wearable sensor of the user.

18. The system according to claim 14, wherein the processor is further configured to communicate the generated prescription to the user through a user interface.

19. The system according to claim 14, wherein the processor is further configured to obtain a feedback from the user in response to the generated prescription.

20. The system according to claim 14, wherein the processor is further configured to generate the prescription by means of a knowledge-based method.

21. A computer-implemented method for health evaluation of a user and prescription comprising iteratively:
receiving overtime health-related information of the user;
monitoring at least one relevant feature representative of a symptomatic event and at least one other relevant feature, being a behavioral event;
establishing a correlation between said at least one relevant feature representative of a symptomatic event and said at least one behavioral event;
detecting in at least one time period of predefined duration an occurrence pattern of the symptomatic event;
using a classifier, determine for the user at least one class associated to at least one type of user presenting a symptom corresponding to a symptomatic event and associated to a recurrent behavior, wherein the classifier receives as input the relevant features representative of the symptomatic event, the at least one behavioral event, the correlation and the occurrence pattern;
generating a prescription of at least one action to be undertaken by the user on the basis of the class so as to improve his health; and
providing said prescription.

22. The method according to claim 21, wherein establishing said correlation from causality detection from the at least one behavioral event to the at least one relevant feature representative of a symptomatic event.

23. The method according to claim 21, wherein the occurrence pattern of the symptomatic event is correlated with contextualized temporal information.

24. A non-transitory computer readable medium comprising instructions which, when executed by a computer, cause the computer to carry out the steps of the method according to claim 21.
